(19) United States
(12) Patent Application Publication
(10) Pub. No.: US 2026/0326512 A1
(43) Pub. Date: Oct. 22, 2026

(54) BLOWER DEVICE

(71) Applicant: MITSUBISHI HEAVY INDUSTRIES THERMAL SYSTEMS, LTD., Tokyo (JP)

(72) Inventor: Noriyuki CHIKAGAWA, Tokyo (JP)

(73) Assignee: MITSUBISHI HEAVY INDUSTRIES THERMAL SYSTEMS, LTD., Tokyo (JP)

(21) Appl. No.: 19/099,661

(22) PCT Filed: Aug. 3, 2023
(86) PCT No.: PCT/JP2023/028464

§ 371 Date: Jan. 29, 2025

(30) Foreign Application Priority Data
Aug. 5, 2022 (JP) 2022-125549

(51) Int. Cl. B60H 3/06; A61L 2/10; A61L 9/20; B60H 1/00

(52) U.S. Cl. CPC B60H 3/06; A61L 2/10; A61L 9/205; B60H 1/00514; A61L 2202/11; A61L 2209/12; A61L 2209/16

(57) ABSTRACT

To simplify the work of attaching an ultraviolet irradiation device. A blower unit comprises: a housing that forms an outer shell; an internal air suction port that is provided inside the housing and suctions internal air into the housing; an external air suction port that is provided to the housing and suctions external air into the housing; an intake damper that selectively closes the internal air suction port or the external air suction port; a filter that is provided inside the housing and captures floating matter floating in the air suctioned into the housing; a first ultraviolet irradiation device that irradiates the filter with an ultraviolet ray; and an attachment part that is fixed to the housing and covers the internal air suction port, and to which the first ultraviolet irradiation device is attached, wherein the first ultraviolet irradiation device overlaps the internal air suction port when the internal air suction port is viewed from above.

### TECHNICAL FIELD

The present disclosure relates to a blower device.

### BACKGROUND ART

A vehicular air conditioning device for adjusting a temperature of air in a vehicle is provided inside a dashboard of the automobile. The vehicular air conditioning device includes a heating ventilation and air conditioning (HVAC) unit that adjusts a temperature of air outside a vehicle (hereinafter, referred to as “external air”) or air inside a vehicle cabin (hereinafter, referred to as “internal air”) and blows the air into the vehicle cabin, and a blower unit that supplies the external air or the internal air to the HVAC unit.

The blower unit may be provided with a device for inactivating microorganisms and viruses adhering to particles floating in the air by irradiating the flowing air with ultraviolet rays. However, when the particles floating in the air are irradiated with the ultraviolet rays, there is a possibility that the microorganisms and the viruses cannot be suitably inactivated unless the air is circulated and the ultraviolet rays are repeatedly irradiated.

In order to solve such a problem, a technique is known in which a filter is provided in a flow path through which air flows, particles are captured by the filter, and the filter is irradiated with ultraviolet rays to perform an inactivation process on microorganisms and viruses (for example, Patent Document 1).

Patent Document 1 describes a vehicular air conditioning device including a blower unit in which an ultraviolet lamp is disposed near a rotation shaft as a light emitting unit that emits light having a wavelength that causes photoexcitation of a photocatalyst at an intake door, and the ultraviolet lamp irradiates a filter with ultraviolet rays.

### CITATION LIST

### Patent Literature

Patent Document 1: JP 2004-291864 A

### SUMMARY OF INVENTION

### Technical Problem

In the configuration described in Patent Document 1, the ultraviolet lamp is disposed at the rotation shaft of the intake door. The rotation shaft of the intake door rotates when the intake door selectively opens and closes an internal air circulation passage and an external air introduction passage. As described above, in a case where a component (for example, the ultraviolet lamp or the like) that needs to be connected to a power source or the like by wiring is provided at the rotating body that performs rotational movement, there is a possibility that routing of the wiring becomes complicated. For this reason, there is a possibility that the work of attaching becomes complicated.

The present disclosure has been made in view of such circumstances, and it is an object of the present disclosure to provide a blower device that can simplify the work of attaching an ultraviolet irradiation device.

### Solution to Problem

In order to solve the problem described above, a blower device of the present disclosure adopts the following devices and parts.

According to an aspect of the present disclosure, there is provided a blower device that supplies external air outside a vehicle or internal air inside a vehicle cabin to a temperature adjustment device, the temperature adjustment device being configured to adjust a temperature of the external air or the internal air and then blow out the external air or the internal air into the vehicle cabin, the blower device including: a housing that forms an outer shell; an internal air suction port that is provided inside the housing and suctions internal air into the housing; an external air suction port that is provided to the housing and suctions external air into the housing; an intake damper that selectively closes the internal air suction port or the external air suction port; a filter that is provided inside the housing and captures floating matter floating in the air suctioned into the housing; a first ultraviolet irradiation device that irradiates the filter with an ultraviolet ray; and an attachment part that is fixed to the housing and covers the internal air suction port and to which the first ultraviolet irradiation device is attached, wherein the first ultraviolet irradiation device overlaps the internal air suction port when the internal air suction port is viewed from above.

### Advantageous Effects of Invention

According to the present disclosure, it is possible to simplify the work of attaching an ultraviolet irradiation device.

### BRIEF DESCRIPTION OF DRAWINGS

FIG. 1 is a vertical cross-sectional view illustrating a blower unit according to an embodiment of the present disclosure.

FIG. 2 is a cross-sectional view taken along line A-A with arrows in FIG. 1.

FIG. 3 is a cross-sectional view taken along line B-B with arrows in FIG. 2.

FIG. 4 is a cross-sectional view illustrating a modification example of FIG. 2.

### DESCRIPTION OF EMBODIMENTS

An embodiment of a blower device (blower unit) according to the present disclosure will be described below with reference to the drawings. In the following description, an up-down direction is referred to as a Z-axis direction, one direction of directions that are orthogonal to the Z-axis direction is referred to as an X-axis direction, and a direction orthogonal to the Z-axis direction and the X-axis direction is referred to as a Y-axis direction.

The blower device according to the present embodiment is adopted to a vehicular air conditioning device that is provided in a space defined by an instrument panel of an automobile, for example. In the following description, the blower device is referred to as a “blower unit”.

The vehicular air conditioning device includes a temperature adjustment device (heating ventilation and air conditioning (HVAC) unit) that controls a temperature of air outside a vehicle (referred to as external air) or air inside a vehicle cabin (referred to as internal air) and blows the air into the vehicle cabin, and a blower unit 10 that supplies the external air or the internal air to the HVAC unit. The HVAC unit is not illustrated.

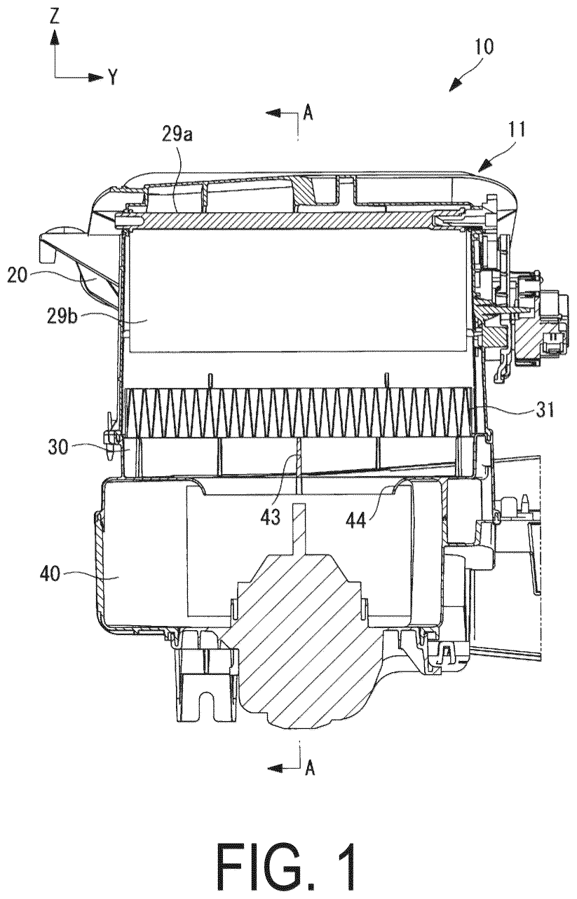

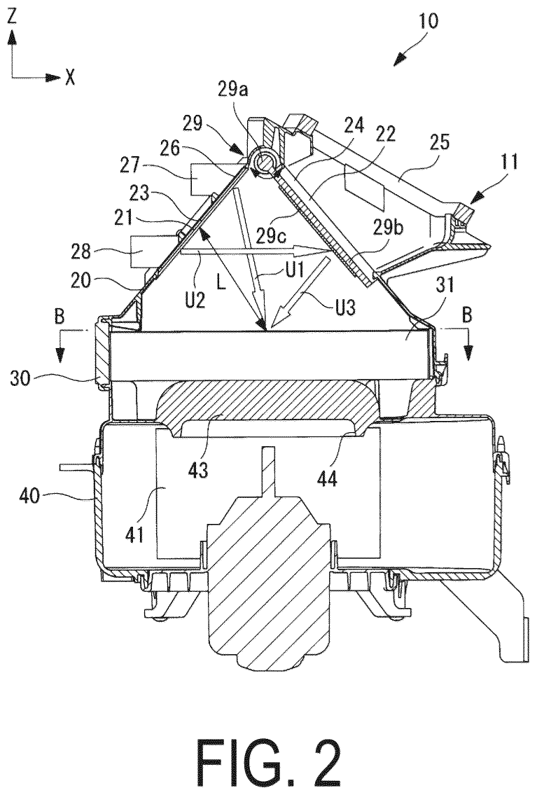

As illustrated in FIG. 1 and FIG. 2, the blower unit 10 includes a housing 11 serving as an outer shell. An air flow path through which the external air or the internal air flows is formed inside the housing 11. The housing 11 respectively suctions the internal air or the external air thereinto from an internal air suction port 23 or an external air suction port 24 formed at an upper end thereof, and respectively suctions the internal air or the external air from a fan suction port 44 formed at a lower end thereof.

As illustrated in FIG. 2, the housing 11 includes an inclined part 20 provided at the upper part thereof, a filter accommodating part 30 that is connected to a lower end of the inclined part 20 and inside which a filter 31 is accommodated, and a fan accommodating part 40 that is connected to a lower end of the filter accommodating part 30 and inside which a fan 41 is accommodated.

The inclined part 20 includes an internal air suction part 21 having a plate shape and extending obliquely downward from a center in the X-axis direction toward one side in the X-axis direction and an external air suction part 22 having a plate shape and extending obliquely downward from the center in the X-axis direction toward the other side in the X-axis direction. Upper ends of the internal air suction part 21 and the external air suction part 22 are connected to each other and inner ends thereof are connected to each other.

An internal air suction port 23 is formed at the internal air suction part 21. The internal air suction port 23 connects an external space and an internal space of the housing 11. An internal air suction duct (not illustrated) is connected to the internal air suction port 23. An upstream end of the internal air suction duct communicates with a space inside the vehicle cabin. The internal air suctioned from the internal air suction duct is suctioned into the housing 11 through the internal air suction port 23.

The internal air suction port 23 is provided with a lattice part (attachment part) 26. The lattice part 26 covers the internal air suction port 23 from the outside. The lattice part 26 includes a plate-like member extending in the X-axis direction along the internal air suction port 23 and a plate-like member extending in the Y-axis direction. The lattice part 26 is fixed to the housing 11 (the internal air suction part 21).

A first ultraviolet irradiation device 27 and a second ultraviolet irradiation device 28 are attached to the lattice part 26. The first ultraviolet irradiation device 27 and the second ultraviolet irradiation device 28 are disposed at positions overlapping the internal air suction port 23 when the internal air suction port 23 is viewed from above.

The first ultraviolet irradiation device 27 is provided above the second ultraviolet irradiation device 28. The first ultraviolet irradiation device 27 is provided above the center of the inclined part 20 in the Z-axis direction. The second ultraviolet irradiation device 28 is provided below the center of the inclined part 20 in the Z-axis direction.

The first ultraviolet irradiation device 27 is provided closer to the center (the center of the inclined part 20) in the X-axis direction than the second ultraviolet irradiation device 28. The first ultraviolet irradiation device 27 is provided on the other side (right side of the page in FIG. 2) relative to the center in the X-axis direction of the internal air suction part 21. The second ultraviolet irradiation device 28 is provided on one side (left side of the page in FIG. 2) relative to the center in the X-axis direction of the internal air suction part 21.

The first ultraviolet irradiation device 27 and the second ultraviolet irradiation device 28 radiate ultraviolet rays. The first ultraviolet irradiation device 27 and the second ultraviolet irradiation device 28 radiate ultraviolet rays to sterilize and inactivate viruses and microorganisms adhering to particles or mist floating in the air flowing inside the housing, and thus purify the air. In the present embodiment, the first ultraviolet irradiation device 27 and the second ultraviolet irradiation device 28 irradiate a filter 31, which will be described later, with ultraviolet rays (specifically, the second ultraviolet irradiation device 28 irradiates the filter 31 with ultraviolet rays via a reflection plate (reflection part) 29c). Thus, the first ultraviolet irradiation device 27 and the second ultraviolet irradiation device 28 mainly sterilize and inactivate the viruses and the microorganisms adhering to the particles and the mist captured by the filter 31.

The first ultraviolet irradiation device 27 and the second ultraviolet irradiation device 28 are not limited as long as these devices radiate ultraviolet rays. In the present embodiment, the first ultraviolet irradiation device 27 and the second ultraviolet irradiation device 28 may have a certain degree of directivity in radiation of ultraviolet rays, such as a device in which an LED that radiates ultraviolet rays is attached to a substrate.

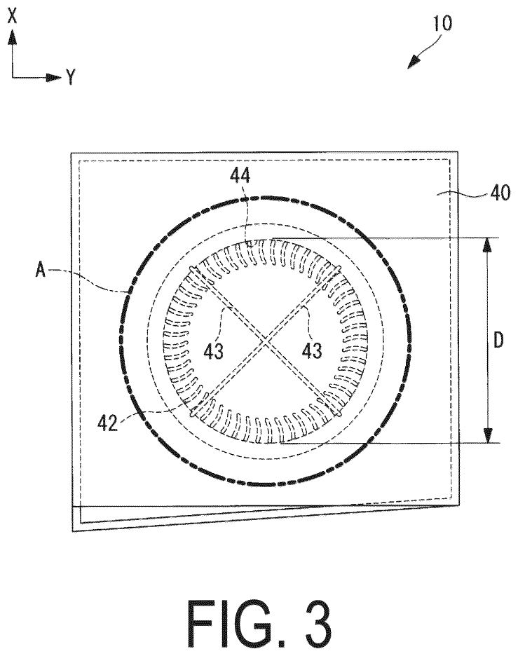

The first ultraviolet irradiation device 27 irradiates the filter 31 with ultraviolet rays as indicated by an arrow Ul in FIG. 2. Specifically, the first ultraviolet irradiation device 27 irradiates a predetermined region A (see FIG. 3) of the filter 31 with ultraviolet rays. The predetermined region A is a region through which a primary flow of the air in the housing 11 passes. Specifically, as indicated by a long-dashed double-dotted line in FIG. 3, the predetermined region A includes a region overlapping with the fan suction port 44 having a bell mouth shape when the filter 31 is viewed from above.

In addition, the second ultraviolet irradiation device 28 irradiates the reflection plate 29c, which will be described later, with ultraviolet rays as indicated by an arrow U2 in FIG. 2. The predetermined region A of the filter 31 is irradiated with some of the ultraviolet rays reflected by the reflection plate 29c as indicated by an arrow U3.

Note that although the ultraviolet rays radiated from the first ultraviolet irradiation device 27 and the ultraviolet rays radiated from the second ultraviolet irradiation device 28 are individually indicated by one arrow in FIG. 2, the ultraviolet rays are actually irradiated with a spread of about 60 degrees in each direction with the direction indicated by the arrow serving as the center. In the first ultraviolet irradiation device 27 and the second ultraviolet irradiation device 28, an amount of ultraviolet rays decreases as a distance from the center of the ultraviolet radiation (the direction indicated by the arrow) increases, but the amount of ultraviolet rays hardly decreases as long as a position is within about 30 degrees from the center.

In the present embodiment, a distance L from the center of the internal air suction port 23 to the center of the filter 31 is substantially 100 mm. Additionally, a diameter D (see FIG. 3) of a range above the bell mouth through which much wind passes is equal to or larger than 200 mm and equal to or smaller than 250 mm. Thus, when the first ultraviolet irradiation device 27 irradiates the center of the filter 31 with the ultraviolet rays, the above-described predetermined region A can be included within the range of 30 degrees from the center. This makes it possible to more effectively irradiate, with the ultraviolet rays, the predetermined region A in which the particles and the like are easily captured.

The external air suction port 24 is formed at the external air suction part 22. The external air suction port 24 connects the external space and the internal space of the housing 11. An external air suction duct 25 that suctions the external air is connected to the external air suction port 24. The external air suctioned from the external air suction duct 25 is suctioned into the housing 11 through the external air suction port 24.

Additionally, an intake damper 29 is provided on the inner side of the inclined part 20. The intake damper 29 is provided at a lower surface of a connecting portion between the internal air suction part 21 and the external air suction part 22.

The intake damper 29 includes a rotation shaft part 29a having a cylindrical shape and extending along the Y-axis direction and a closing part 29b having a plate shape and having one end fixed to the rotation shaft part 29a. The rotation shaft part 29a is fixed to the lower surface of the connecting portion between the internal air suction part 21 and the external air suction part 22 so as to be rotatable by a predetermined angle about a center axis extending along the Y-axis direction. The closing part 29b is larger than the internal air suction port 23 and the external air suction port 24, and is capable of closing the internal air suction port 23 and the external air suction port 24.

In the intake damper 29, when the rotation shaft part 29a is rotated manually or by a driving force from a drive device, the closing part 29b moves according to the rotation of the rotation shaft part 29a. Specifically, the closing part 29b moves between a position where the internal air suction port 23 is covered and closed from the inside and a position where the external air suction port 24 is covered and closed from the inside. That is, the intake damper 29 can switch between a state where the internal air suction port 23 is closed and a state where the external air suction port 24 is closed.

The reflection plate 29c is provided at one surface of the closing part 29b (a surface facing the internal space of the housing 11 in a state where the intake damper 29 closes the external air suction port 24). The reflection plate 29c is made of a material (for example, aluminum) capable of reflecting ultraviolet rays. The reflection plate 29c is a flat plate-shaped member. The reflection plate 29c is attached to one surface of the closing part 29b so as to cover substantially the entire region of the one surface. The reflection plate 29c is provided so as to face the filter 31 in a state where the intake damper 29 closes the external air suction port 24.

As illustrated in FIG. 3, the filter accommodating part 30 is a member having a rectangular frame shape in top view. As illustrated in FIG. 2, an upper end of the filter accommodating part 30 is connected to a lower end of the inclined part 20, and a lower end thereof is connected to an upper end of the fan accommodating part 40.

As illustrated in FIG. 3, the filter 31 is accommodated inside the filter accommodating part 30 over substantially the entire region in the X-axis direction and the Y-axis direction. The filter 31 is provided so as to cover substantially the entire region of a cross section of the flow path.

The filter 31 is provided in the filter accommodating part 30. The filter 31 is provided on a downstream side of the first ultraviolet irradiation device 27. When the air (internal air or external air) flowing inside the housing 11 passes through the filter 31, the filter 31 captures particles or mist floating in the air. The filter 31 may be made of a non-woven fabric or may be made of a material having photocatalytic properties. Note that in the blower unit 10 of the present embodiment, viruses and the like are inactivated by irradiating the filter 31 with ultraviolet rays. Thus, an expensive filter such as a photocatalyst filter does not need to be used, and even an inexpensive filter such as a filter made of a non-woven fabric or the like can sufficiently purify the air. Thus, when the inexpensive filter such as the non-woven fabric is used, the cost can be reduced while the effect of sufficiently purifying the air is obtained.

Additionally, as illustrated in FIG. 1 and FIG. 2, the filter 31 has a plurality of pleats. That is, in the filter 31, mountain-shaped portions and valley-shaped portions extending in the X-axis direction are alternately aligned along the Y-axis direction.

In the present embodiment, as illustrated in FIG. 2, the first ultraviolet irradiation device 27 radiates ultraviolet rays in the X-axis direction. In this way, radiating the ultraviolet rays from the same direction as the extending direction of the mountain-shaped portions causes the mountain-shaped portions to be less likely to block the ultraviolet rays. Thus, the valley-shaped portions can also be suitably irradiated with the ultraviolet rays.

The fan accommodating part 40 is a member having a circular shape in top view and accommodates the fan 41 therein as illustrated in FIG. 1 and FIG. 2. The fan suction port 44 is formed at a bottom surface part of the fan accommodating part 40. The fan suction port 44 allows the internal space and the external space of the housing 11 to communicate with each other.

As illustrated in FIG. 3, the fan 41 includes a plurality of blades 42 aligned at equal intervals along an edge of the fan suction port 44. When the fan 41 is driven, the plurality of blades 42 are rotated to suction internal air or external air into the housing 11 through the internal air suction port 23 or the external air suction port 24.

The fan accommodating part 40 is provided with reinforcing members 43 each of which has a plate shape so as to straddle the fan suction port 44. The two reinforcing members 43 are arranged so as to form a cross as viewed from above, and prevent the filter from being suctioned.

In addition, the blower unit 10 includes a control unit (not illustrated). The control unit controls the intake damper 29, and thus, can switch between a state where the intake damper 29 closes the internal air suction port 23 and a state where the intake damper 29 closes the external air suction port 24. In addition, the control unit controls the start and stop of the radiation from the first ultraviolet irradiation device 27 and the second ultraviolet irradiation device 28.

The control unit has an internal air mode and an external air mode. The internal air mode is a mode in which the air inside the vehicle cabin (internal air) is suctioned, and a temperature of the suctioned air is adjusted and then the adjusted air is discharged into the vehicle cabin. Additionally, the external air mode is a mode in which the air outside the vehicle (external air) is suctioned, a temperature of the suctioned air is adjusted and then the adjusted air is discharged into the vehicle cabin.

The control unit controls the first ultraviolet irradiation device 27 and the second ultraviolet irradiation device 28 so as to stop the radiation of the ultraviolet rays in a state where the intake damper 29 closes the internal air suction port 23 in the external air mode. In addition, in the internal air mode, the control unit controls the first ultraviolet irradiation device 27 and the second ultraviolet irradiation device 28 so as to radiate the ultraviolet rays in a state in which the intake damper 29 closes the external air suction port 24.

The control unit (controller) includes, for example, a central processing unit (CPU) (processor), a main memory (primary storage device), a secondary storage (memory), and the like. Further, the control unit may include a communication unit for transmitting and receiving information to and from another device.

The main memory includes a writable memory such as a cache memory and a random access memory (RAM) and is used as a work area for reading an execution program of the CPU and writing processing data by the execution program, for example.

The secondary storage is a non-transitory computer-readable storage medium. Examples of the secondary storage include a magnetic disk, a magneto-optical disk, a CD-ROM, a DVD-ROM, and a semiconductor memory.

A series of processing operations for implementing various functions is, for example, stored in the secondary storage in the form of a program, and the CPU reads the program into the main memory and executes information processing and computation processing, thereby implementing various functions. The program may be a program pre-installed in the secondary storage, a program provided in a state of being stored in a computer-readable storage medium, a program distributed through wired or wireless communication means, or the like. The computer-readable storage medium includes a magnetic disk, a magneto-optical disk, a CD-ROM, a DVD-ROM, and a semiconductor memory.

Next, an air flow in an air conditioner and the like will be described with reference to FIG. 2. Note that FIG. 2 illustrates a state in which the intake damper 29 closes the external air suction port 24 and opens the internal air suction port 23 (that is, a state in the internal air mode).

### Internal Air Mode

First, an air flow and the like in the internal air mode will be described.

In the internal air mode, the control unit controls the intake damper 29 such that the closing part 29b closes the external air suction port 24. Thus, the internal air suction port 23 is opened, so that the internal air (air in the vehicle cabin) is suctioned into the housing 11 through the internal air suction port 23.

The internal air suctioned into the housing 11 flows from the upper side to the lower side in the housing 11. In the flow, the internal air passes through the filter 31. At this time, since the primary flow of the internal air flows through a route having a small pressure loss, the primary flow passes through the predetermined region A of the filter 31. Particles and the like floating in the internal air are captured by the filter 31 while passing through the filter 31. Since a large amount of internal air passes through the predetermined region A of the filter 31, a larger amount of particles and the like are captured in the predetermined region A than those in the other regions.

The internal air purified by passing through the filter 31 is discharged from the fan suction port 44 to the outside of the housing 11, is supplied to the HVAC unit through a duct (not illustrated) or the like, and is adjusted in temperature by the HVAC unit. The internal air adjusted in temperature is blown into the vehicle cabin.

In the internal air mode, the control unit causes the first ultraviolet irradiation device 27 and the second ultraviolet irradiation device 28 to radiate the ultraviolet rays. The first ultraviolet irradiation device 27 irradiates the predetermined region A of the filter 31 with the ultraviolet rays (see the arrow U1 in FIG. 2). This inactivates viruses and the like adhering to the particles and the like captured by the filter 31 (in particular, the predetermined region A).

In addition, the second ultraviolet irradiation device 28 irradiates the reflection plate 29c with the ultraviolet rays as indicated by the arrow U2 in FIG. 2. Some of the ultraviolet rays reflected by the reflection plate 29c are irradiated toward the predetermined region A of the filter 31 as indicated by the arrow U3. This inactivates viruses and the like adhering to the particles and the like captured by the filter 31 (in particular, the predetermined region A).

### External Air Mode

First, an air flow and the like in the external air mode will be described.

In the internal air mode, the control unit controls the intake damper 29 such that the closing part 29b closes the internal air suction port 23. Thus, the external air suction port 24 is opened, so that the external air (air outside the vehicle) is suctioned into the housing 11 through the external air suction port 24.

Since a flow of the external air suctioned into the housing 11 is the same as the flow of the internal air in the housing 11, detailed description thereof will be omitted.

The ultraviolet irradiation device overlaps the internal air suction port 23 when the internal air suction port 23 is viewed from above. Thus, in the external air mode in which the intake damper 29 closes the internal air suction port 23, even when ultraviolet rays are radiated from the first ultraviolet irradiation device 27 and the second ultraviolet irradiation device 28, the ultraviolet rays are blocked by the closing part 29b of the intake damper 29 and do not reach the filter 31. In addition, in the external air mode, since the inside of the vehicle can be ventilated, irradiation of ultraviolet rays is not necessary. Due to this, in the external air mode, the control unit does not cause the first ultraviolet irradiation device 27 and the second ultraviolet irradiation device 28 to radiate ultraviolet rays.

According to the present embodiment, the following operational effects are obtained.

In the present embodiment, the first ultraviolet irradiation device 27 and the second ultraviolet irradiation device 28 irradiate the filter 31 with ultraviolet rays. This makes it possible to inactivate microorganisms, viruses, and the like adhering to floating matter captured by the filter 31.

Further, in the present embodiment, the first ultraviolet irradiation device 27 and the second ultraviolet irradiation device 28 are provided at a covering part that is a stationary object fixed to the housing 11. This simplifies routing of wiring or the like in a case where the first ultraviolet irradiation device 27 and the second ultraviolet irradiation device 28 are connected to another device (for example, a power source or the like) by wiring, as compared with a case where the first ultraviolet irradiation device 27 and the second ultraviolet irradiation device 28 are provided at a moving body such as a rotating body. Thus, the work of attaching the first ultraviolet irradiation device 27 and the second ultraviolet irradiation device 28 can be simplified.

When the internal air is suctioned by the blower unit 10 and the internal air adjusted in temperature is blown into the vehicle cabin (that is, when the internal air is circulated), since the volume of the vehicle cabin is small, the concentration of viruses and the like increases, and the air is easily contaminated.

In the present embodiment, the control unit controls the first ultraviolet irradiation device 27 and the second ultraviolet irradiation device 28 so as to radiate ultraviolet rays in a state where the intake damper 29 opens the internal air suction port 23. Thus, in the case of circulating the internal air in which the air is easily contaminated, the virus or the like adhering to the floating matter captured by the filter 31 can be inactivated. As a result, the air blown into the vehicle cabin can be purified.

In addition, in the present embodiment, the first ultraviolet irradiation device 27 and the second ultraviolet irradiation device 28 are provided so as to overlap the internal air suction port 23 when the internal air suction port 23 is viewed from above. Thus, in a state where the intake damper 29 closes the internal air suction port 23, even when ultraviolet rays are radiated, the ultraviolet rays are blocked by the intake damper 29 and do not reach the filter 31. In the present embodiment, the control unit stops the radiation of the ultraviolet rays in a state where the intake damper 29 closes the internal air suction port 23, thereby allowing unnecessary radiation of the ultraviolet rays to be suppressed. As a result, energy can be saved, and energy exchange efficiency can be improved.

In the present embodiment, the first ultraviolet irradiation device 27 is provided at the lattice part 26 provided so as to cover the internal air suction port 23. Thus, the ultraviolet rays radiated from the first ultraviolet irradiation device 27 are strongly irradiated to a region of the filter 31 on the internal air suction port 23 side, which is close to the first ultraviolet irradiation device 27. On the other hand, a region of the filter 31 on the external air suction port 24 side, which is relatively far from the first ultraviolet irradiation device 27, is weakly irradiated with the ultraviolet rays.

On the other hand, in the present embodiment, the reflection plate 29c is opposed to the filter 31 in a state where the intake damper 29 closes the external air suction port 24. Accordingly, the reflection plate 29c can reflect, to the filter 31, the ultraviolet rays irradiated from the second ultraviolet irradiation device 28. The ultraviolet rays reflected by the reflection plate 29c are strongly irradiated to a region of the filter 31 on the external air suction port 24 side, which is close to the reflection plate 29c. On the other hand, a region of the filter 31 on the internal air suction port 23 side, which is relatively far from the reflection plate 29c, is weakly irradiated with the ultraviolet rays.

As described above, although the ultraviolet rays from the first ultraviolet irradiation device 27 and the second ultraviolet irradiation device 28 are strongly irradiated to the region of the filter 31 on the internal air suction port 23 side, the ultraviolet rays from the first ultraviolet irradiation device 27 and the second ultraviolet irradiation device 28 reflected by the reflection plate 29c are weakly irradiated to the region of the filter 31. On the other hand, although the ultraviolet rays from the first ultraviolet irradiation device 27 and the second ultraviolet irradiation device 28 are weakly irradiated to the region of the filter 31 on the external air suction port 24 side, the ultraviolet rays from the first ultraviolet irradiation device 27 and the second ultraviolet irradiation device 28 reflected by the reflection plate 29c are strongly irradiated to the region of the filter 31. Thus, it is possible to reduce variations in intensity of the ultraviolet rays with which the filter 31 is irradiated.

Further, in the present embodiment, the first ultraviolet irradiation device 27 disposed above irradiates the filter 31 with the ultraviolet rays. The first ultraviolet irradiation device 27 is disposed above (that is, disposed away from the filter 31). For this reason, the ultraviolet rays radiated from the first ultraviolet irradiation device 27 easily spread before reaching the filter 31. Thus, compared to, for example, a structure in which the filter 31 is irradiated with ultraviolet rays by the second ultraviolet irradiation device 28, a wide range of the filter 31 can be irradiated with ultraviolet rays.

### MODIFICATION EXAMPLE

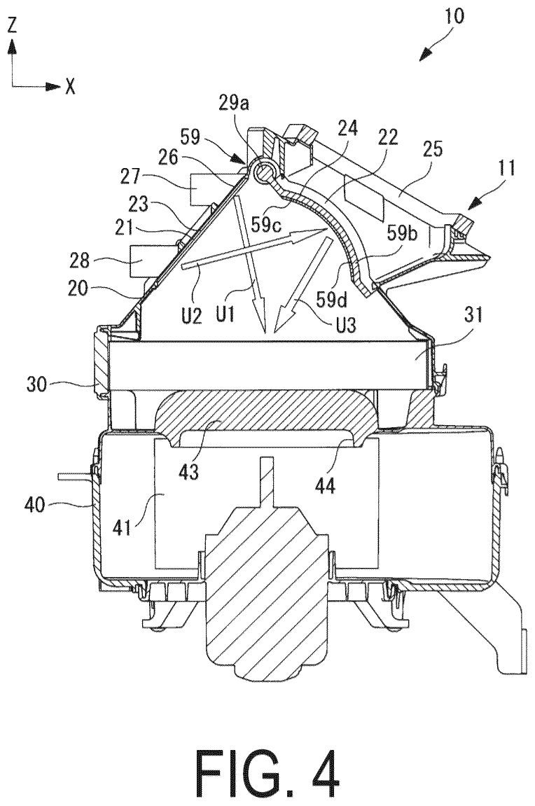

Next, a modification example of the embodiment described above will be described using FIG. 4.

In this modification example, a shape of the closing part of the intake damper and a shape of the reflection plate are different from those of the above-described embodiment. The others are similar to those of the above-described embodiment, so that similar configurations are denoted by the same reference signs, and detailed descriptions thereof will be omitted.

A closing part 59b of an intake damper 59 according to the present modification example is recessed in a center region. To be more specific, one surface of the closing part 59b (a surface facing the internal space of the housing 11 in a state where the intake damper 29 closes the external air suction port 24) is formed as a recessed part 59d recessed in a spherical shape.

Note that a region other than the center region of the closing part 59b (other than the recessed part 59d) is formed in a flat plate shape. With this configuration, when the internal air suction port 23 and the external air suction port 24 are closed, the internal air suction port 23 and the external air suction port 24 can be brought into surface contact with the edges of the respective openings, resulting in effectively closing the internal air suction port 23 and the external air suction port 24.

In addition, a reflection plate 59c according to the present modification example is curved so as to correspond to the shape of the recessed part 59d. Specifically, one surface (a surface facing the internal space of the housing 11) is curved so as to be recessed in a spherical shape. The reflection plate 59c is attached to the recessed part 59d so as to cover the entire region of the recessed part 59d. The reflection plate 59c is recessed such that the reflected ultraviolet rays are concentrated on the filter 31 when the reflection plate 59c reflects the ultraviolet rays radiated from the second ultraviolet irradiation device 28.

According to the present modification example, the following operational effects are obtained.

In the present modification example, the reflection plate 59c is recessed such that the reflected ultraviolet rays are concentrated on the filter 31. Thus, the filter 31 can be irradiated with a larger amount of ultraviolet rays, thereby allowing viruses or the like adhering to floating matter captured by the filter 31 to be more suitably inactivated. As a result, the air blown into the vehicle cabin can be purified.

Note that the present disclosure is not limited to the respective embodiments described above and can be modified as appropriate without departing from the gist of the present disclosure.

For example, in the embodiment described above, the example in which two ultraviolet irradiation devices, that is, the first ultraviolet irradiation device 27 and the second ultraviolet irradiation device 28, are provided has been described. However, the present disclosure is not limited thereto. Only either the first ultraviolet irradiation device 27 or the second ultraviolet irradiation device 28 may be provided. Further, a plurality of first ultraviolet irradiation devices 27 may be provided, and a plurality of second ultraviolet irradiation devices 28 may be provided.

Moreover, in the embodiment described above, the example has been described in which the control device automatically causes the first ultraviolet irradiation device 27 and the second ultraviolet irradiation device 28 to be started and stopped. However, for example, in a case where the blower unit of the present disclosure is provided in an inexpensive vehicle in accordance with manual operation specifications or the like, the first ultraviolet irradiation device 27 and the second ultraviolet irradiation device 28 may be started and stopped by using a limit switch.

The blower device according to the embodiment described above is understood as follows, for example.

According to a first aspect of the present disclosure, there is provided a blower device (10) that supplies external air outside a vehicle or internal air inside a vehicle cabin to a temperature adjustment device, the temperature adjustment device being configured to adjust a temperature of the external air or the internal air and then blow out the external air or the internal air into the vehicle cabin, the blower device including: a housing (11) that forms an outer shell; an internal air suction port (23) that is provided inside the housing and suctions internal air into the housing; an external air suction port (24) that is provided to the housing and suctions external air into the housing; an intake damper (29) that selectively closes the internal air suction port or the external air suction port; a filter (31) that is provided inside the housing and captures floating matter floating in the air suctioned into the housing; a first ultraviolet irradiation device (27) that irradiates the filter with an ultraviolet ray, and an attachment part (26) that is fixed to the housing and covers the internal air suction port, and to which the first ultraviolet irradiation device is attached, wherein the first ultraviolet irradiation device overlaps the internal air suction port when the internal air suction port is viewed from above.

In the configuration described above, the first ultraviolet irradiation device irradiates the filter with the ultraviolet rays. This makes it possible to inactivate microorganisms, viruses, and the like adhering to the floating matter captured by the filter.

In addition, in the above-described configuration, the first ultraviolet irradiation device is provided at a covering part that is a stationary object fixed to the housing. This can simplify routing of wiring or the like in a case where the first ultraviolet irradiation device is connected to another device (for example, a power source or the like) by the wiring, as compared with a case where the first ultraviolet irradiation device is provided at a moving body such as a rotating body. Thus, the work of attaching the first ultraviolet irradiation device can be simplified.

Note that “cover the internal air suction port” does not mean a state of covering the entire region of the internal air suction port, but means a state where a member having a shape (for example, a lattice shape) that does not hinder the suction of the internal air through the internal air suction port overlaps the internal air suction port in the vicinity of the internal air suction port.

Further, according to a second aspect of the present disclosure, there is provided a blower device, in the blower device according to the first aspect, further including: a control unit that controls the first ultraviolet irradiation device to stop the irradiation of the ultraviolet ray in a state where the intake damper closes the internal air suction port and to perform the irradiation of the ultraviolet ray in a state where the intake damper closes the external air suction port.

When the internal air is suctioned by the blower device and the internal air adjusted in temperature is blown into the vehicle cabin (that is, when the internal air is circulated), since the volume of the vehicle cabin is small, the concentration of viruses and the like increases, and the air is easily contaminated.

In the above-described configuration, the control unit controls the first ultraviolet irradiation device to radiate ultraviolet rays in a state where the intake damper opens the internal air suction port. Accordingly, in a case where the internal air that is easily contaminated is circulated, the viruses or the like adhering to the floating matter captured by the filter can be inactivated. As a result, the air blown into the vehicle cabin can be purified.

In addition, in the above-described configuration, the first ultraviolet irradiation device overlaps the internal air suction port when the internal air suction port is viewed from above. Thus, in a state in which the intake damper closes the internal air suction port, even when ultraviolet rays are radiated, the ultraviolet rays are blocked by the intake damper and does not reach the filter. In the above-described configuration, since the control unit stops the irradiation of the ultraviolet rays in a state where the intake damper closes the internal air suction port, unnecessary irradiation of the ultraviolet rays can be suppressed. As a result, energy can be saved, and energy exchange efficiency can be improved.

Further, according to a third aspect of the present disclosure, there is provided a blower device, in the blower device according to the first aspect or the second aspect, further including: a reflection part (29c) that reflects the ultraviolet ray; and a second ultraviolet irradiation device (28) that irradiates the reflection part with an ultraviolet ray, wherein the reflection part is provided at a surface of the intake damper that faces the inside of the housing at a timing when the intake damper is closing the external air suction port, and is opposed to the filter in a state where the intake damper closes the external air suction port.

In the above-described configuration, the first ultraviolet irradiation device is provided at the attachment part attached so as to cover the internal air suction port. Thus, the ultraviolet rays radiated from the first ultraviolet irradiation device is strongly irradiated to a region of the filter on the internal air suction port side, which is close to the first ultraviolet irradiation device. On the other hand, a region of the filter on the external air suction port side, which is relatively far from the first ultraviolet irradiation device, is weakly irradiated with the ultraviolet rays.

On the other hand, in the above-described configuration, the reflection part is opposed to the filter in a state where the intake damper closes the external air suction port. With this configuration, the reflection part can reflect, to the filter, the ultraviolet rays radiated from the second ultraviolet irradiation device. The ultraviolet rays reflected by the reflection part is strongly irradiated to a region of the filter on the external air suction port side, which is close to the reflection part. On the other hand, a region of the filter on the internal air suction port side, which is relatively far from the reflection part, is weakly irradiated with the ultraviolet rays.

As described above, the region of the filter on the internal air suction port side is strongly irradiated with the ultraviolet rays radiated from the first ultraviolet irradiation device, but weakly irradiated with the ultraviolet rays that have been radiated from the second ultraviolet irradiation device and that have been reflected by the reflection part. On the other hand, the region of the filter on the external air suction port side is weakly irradiated with the ultraviolet rays radiated from the first ultraviolet irradiation device, but is strongly irradiated with the ultraviolet rays that have been radiated from the second ultraviolet irradiation device and that have been reflected by the reflection part. Thus, variations in intensity of the ultraviolet rays with which the filter is irradiated can be reduced.

Further, according to a fourth aspect of the present disclosure, in the blower device according to the third aspect, the reflection part is recessed, and thus the reflected ultraviolet ray is concentrated on the filter in the state where the intake damper closes the external air suction port.

In the above-described configuration, the reflection part is recessed such that the reflected ultraviolet rays are concentrated on the filter. Accordingly, the filter is irradiated with more ultraviolet rays, resulting in more suitably inactivating the viruses or the like adhering to the floating matter captured by the filter. As a result, the air blown into the vehicle cabin can be purified.

### REFERENCE SIGNS LIST

10: Blower unit
11: Housing
20: Inclined part
21: Internal air suction part
22: External air suction part
23: Internal air suction port
24: External air suction port
25: External air suction duct
26: Lattice part (attachment part)
27: First ultraviolet irradiation device
28: Second ultraviolet irradiation device
29: Intake damper
29a: Rotation shaft part
29b: Closing part
29c: Reflection plate (reflection part)
30: Filter accommodating part
31: Filter
40: Fan accommodating part
41: Fan
42: Blade
43: Reinforcing member
44: Fan suction port
59: Intake damper
59b: Closing part
59c: Reflection plate
59d: Recessed part

1. A blower device that supplies external air outside a vehicle or internal air inside a vehicle cabin to a temperature adjustment device, the temperature adjustment device being configured to adjust a temperature of the external air or the internal air and then blow out the external air or the internal air into the vehicle cabin, the blower device comprising:
a housing that forms an outer shell;
an internal air suction port that is provided inside the housing and suctions internal air into the housing;
an external air suction port that is provided to the housing and suctions external air into the housing;
an intake damper that selectively closes the internal air suction port or the external air suction port;
a filter that is provided inside the housing and captures floating matter floating in the air suctioned into the housing;
a first ultraviolet irradiation device that irradiates the filter with an ultraviolet ray; and
an attachment part that is fixed to the housing and covers the internal air suction port and to which the first ultraviolet irradiation device is attached, wherein
the first ultraviolet irradiation device overlaps the internal air suction port when the internal air suction port is viewed from above.

2. The blower device according to claim 1, further comprising:
a control unit that controls the first ultraviolet irradiation device to stop the irradiation of the ultraviolet ray in a state where the intake damper closes the internal air suction port and to perform the irradiation of the ultraviolet ray in a state where the intake damper closes the external air suction port.

3. The blower device according to claim 1, further comprising:
a reflection part that reflects the ultraviolet ray; and
a second ultraviolet irradiation device that irradiates the reflection part with the ultraviolet ray, wherein
the reflection part is provided at a surface of the intake damper that faces the inside of the housing at a timing when the intake damper is closing the external air suction port, and is opposed to the filter in a state where the intake damper closes the external air suction port.

4. The blower device according to claim 3, wherein
the reflection part is recessed, and thus the reflected ultraviolet ray is concentrated on the filter in the state where the intake damper closes the external air suction port.

5. The blower device according to claim 2, further comprising:
a reflection part that reflects the ultraviolet ray; and
a second ultraviolet irradiation device that irradiates the reflection part with the ultraviolet ray, wherein
the reflection part is provided at a surface of the intake damper that faces the inside of the housing at a timing when the intake damper is closing the external air suction port, and is opposed to the filter in a state where the intake damper closes the external air suction port.
